SEQUENCE LISTING (6 sequences)

SEQ ID NO: 1 - protein, 4 aa, Artificial sequence
  Synthetic peptide
RGDS

SEQ ID NO: 2 - protein, 5 aa, Artificial sequence
  Synthetic peptide
IKVAV

SEQ ID NO: 3 - protein, 6 aa, Artificial sequence
  Synthetic peptide
VVAAEE

SEQ ID NO: 4 - protein, 4 aa, Artificial sequence
  Synthetic peptide
VVAA

SEQ ID NO: 5 - protein, 4 aa, Artificial sequence
  Synthetic peptide
AAVV

SEQ ID NO: 6 - protein, 6 aa, Artificial sequence
  Synthetic peptide
AAAVVV

(19) United States
(12) Patent Application Publication
(10) Pub. No.: US 2017/0021056 A1
(43) Pub. Date: Jan. 26, 2017

(54) PEPTIDE AMPHIPHILE BIOMATERIALS FOR NERVE REPAIR

(71) Applicants: Northwestern University, Evanston, IL (US); The Regents of the University of California, Oakland, CA (US)

(72) Inventors: Samuel I. Stupp, Chicago, IL (US); Mark T. McClendon, Evanston, IL (US); Reza Jarrahy, Los Angeles, CA (US); Akishige Hokugo, Los Angeles, CA (US)

(21) Appl. No.: 15/215,301

(22) Filed: Jul. 20, 2016

Related U.S. Application Data
(60) Provisional application No. 62/194,641, filed on Jul. 20, 2015.

(51) Int. Cl. A61L 27/22; A61L 27/54; A61L 27/50

(52) U.S. Cl. CPC A61L 27/22; A61L 27/50; A61L 27/54; A61L 2430/32

(57) ABSTRACT

Provided herein are compositions comprising aligned peptide amphiphile biomaterials, methods of generating such alignment, and their use in nerve repair applications. In particular, peptide amphiphile nanofibers are aligned using a screen extrusion method, and find use in the repair of peripheral nerve injuries.

### CROSS-REFERENCE TO RELATED APPLICATIONS

The present application claims the priority benefit of U.S. Provisional Patent Application 62/194,641, filed Jul. 20, 2015, which is incorporated by reference in its entirety.

### STATEMENT REGARDING FEDERAL FUNDING

This invention was made with government support under R01 DE015920 and R01 EB003806 awarded by the National Institutes of Health. The government has certain rights in the invention.

### FIELD

Provided herein are compositions comprising aligned peptide amphiphile biomaterials, methods of generating such alignment, and their use in nerve repair applications. In particular, peptide amphiphile nanofibers are aligned using a screen extrusion method, and find use in the repair of peripheral nerve injuries.

### BACKGROUND

Peripheral nerve injuries can result in lifelong disability. Primary coaptation is the treatment of choice when the gap between transected nerve ends is short. Long nerve gaps seen in more complex injuries often require autologous nerve grafts or nerve conduits implemented into the repair. Nerve grafts, however, cause morbidity and bioactive loss at donor sites, which are limited in number. Nerve conduits, in turn, lack an internal scaffold to support and guide axonal regeneration, resulting in decreased efficacy over longer nerve gap lengths.

It is estimated that at least 200,000 peripheral nerve repairs are performed annually in the United States (ref. 1; herein incorporated by reference in its entirety). Traumatic peripheral nerve injuries represent a particular challenge for reconstructive surgeons due to their strong association with long-term disability and poor bioactive outcomes. For example, only 7% of patients who sustain a gunshot wound to an extremity with a resultant peripheral nerve injury attain normal limb function, compared to 39% of patients who achieve bioactive recovery in the setting of a gunshot-induced extremity injury without nerve involvement (ref. 2; herein incorporated by reference in its entirety). This poor prognosis is due to the principle of Wallerian degeneration, whereby the nerve segment distal to the site of injury becomes dysfunctional, which in turn leads to atrophy and malfunction of the end organ supplied by the nerve.

Various techniques for nerve repair exist. Direct neurorrhaphy (end-to-end repair) of a transected nerve is an effective means of repair when there is no gap between the cut nerve ends or if that gap is less than 1 cm. This avoids excessive tension over the suture line, which has been shown to negatively affect bioactive outcomes (refs. 3,4; herein incorporated by reference in their entireties). Complex nerve injuries with gaps of greater than 1 cm can be repaired using interposition autologous nerve grafts. Donor nerve, however, is a limited resource. Moreover, its harvest results in a bioactive deficit at the donor site and can be associated with donor-site morbidity, such as pain, infection, or neuroma formation (ref. 5; herein incorporated by reference in its entirety). The use of cadaveric nerve allograft has been shown to be effective in the repair of peripheral nerve defects up to 5 cm in length (ref. 6; herein incorporated by reference in its entirety). One advantage to nerve allografts in longer gaps is their ability to provide an internal structure and extracellular matrix that serve as a scaffold to support directional migration of reparative Schwann cells. Cadaveric nerve, however, may be associated with immunologic responses, including graft vs. host disease, or transmission of human pathogens. Recently, improvements have been made in the processing techniques used to prepare cadaveric nerve, resulting in improved removal of immunogenic elements while preserving fascicular architecture (ref. 7; herein incorporated by reference in its entirety). Currently available collagen and poly(lactic-co-glycolic acid) (PLGA) nerve conduits can be effective in the repair of short segment peripheral nerve injuries. Empty collagen conduits, for example, have reported effective repair lengths ranging from 1 cm (ref. 8; herein incorporated by reference in its entirety) to less than 3 cm (ref. 9; herein incorporated by reference in its entirety). However, as the length of the defect increases, the efficacy of these empty nerve conduits declines, likely because they are unable to mimic the native internal architecture of the nerve and provide no internal scaffolding to support nerve regeneration.

Due to the shortcomings associated with these approaches to nerve repair, the development of bioengineered nerve conduits that can serve as nerve graft substitutes in the setting of complex nerve injury has been an active area of focus in tissue engineering research.

### SUMMARY

Provided herein are compositions comprising aligned peptide amphiphile biomaterials, methods of generating such alignment, and their use in nerve repair applications. In particular, peptide amphiphile nanofibers are aligned using a screen extrusion method, and find use in the repair of peripheral nerve injuries.

In some embodiments, provided herein are compositions comprising peptide amphiphile nanofibers aligned within a conduit. In some embodiments, the conduit comprises a polymer material. In some embodiments, the polymer material is biodegradeable. In some embodiments, the polymer material is selected from polyglycolic acid (PGA), polylactic acid (PLA), poly(lactic acid-co-glycolic acid) (PLGA), poly-ε-caprolactone (PCL), poly(diol citrate), and combinations thereof. In some embodiments, the internal diameter of the conduit is 0.5-3 mm in diameter. In some embodiments, the conduit is 2-30 mm in length. In some embodiments, the peptide amphiphiles comprise a hydrophobic non-peptide tail, a structured peptide segment, a charged peptide segment. In some embodiments, all or a portion of the peptide amphiphiles further comprise a terminal bioactive moiety. In some embodiments, the terminal bioactive moiety is selected from RGDS (SEQ ID NO: 1) and IKVAV (SEQ ID NO: 2). In some embodiments, the peptide amphiphiles comprise the sequence VVAAEE (SEQ ID NO: 3). In some embodiments, the peptide amphiphiles further comprise a palmitoyl hydrophobic non-peptide tail.

In some embodiments, provided herein are methods of aligning peptide amphiphile (PA) nanofibers comprising extruding a PA solution through a size-limiting filter. In some embodiments, the size-limiting filter is a mesh screen. In some embodiments, the size-limiting filter comprises 10-100 μm pores (e.g., 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, and ranges therebetween). In some embodiments, the PA solution is extruded through the mesh screen into a conduit. In some embodiments, the internal diameter of the conduit is 0.5-3 mm in diameter. In some embodiments, the conduit is 2-30 mm in length.

In some embodiments, provided herein are methods of repairing or reconstructing a peripheral nerve defect comprising placing the defect in contact the peptide amphiphiles, aligned nanofibers, and/or conduit/PA constructs described herein.

In some embodiments, provided herein is the use of the peptide amphiphiles, aligned nanofibers, and/or conduit/PA constructs described herein for tissue regeneration.

### DRAWINGS

FIG. 1. The PA solution is loaded into the PLGA tubes by first passing through a 40-mm mesh screen. The liquid crystalline behavior of nanofibers allows them to align in response to the shear flow experienced as they pass through the mesh. After the PA solution fills the PLGA tube the entire implant is submerged in a 20 mM CaCl2 bath, thus trapping the nanofiber alignment in a gel-state. Nanofiber size is exaggerated for graphical appeal.

FIG. 2. Gross images of sciatic nerve during the surgery. (Panel A) Measurement of the 12 mm segment of nerve to be excised. (Panel B) Transection of the sciatic nerve. Arrows indicate the proximal and distal nerve stumps, which are sutured to surrounding adventitia to prevent migration. (Panel C) Resected nerve segment is sutured back in place as an autograft. Arrows indicate the proximal and distal nerve stumps. (Panel D) The proximal and distal nerve segments are bridged using PLGA/backbone-PA or PLGA/RGDS-PA (SEQ ID NO: 1) conduit between the two ends. Arrows indicate the proximal and distal nerve stumps.

FIGS. 3A-3C. FIG. 3A: SEM showing the aligned PA nanofibers inside the PLGA tube constructs. The tubes were cut in half along their long axis to expose the nanofibers. FIG. 3B: ˜0.5 mm longitudinal section of the tube constructs is imaged using optical microscopy. Panel a: Imaged under brightfield mode, the porous scaffold is shown with the inner PA gel on the right. Panel b: Imaged between cross polars with orientation shown in the upper left, domains of nanofibers aligned diagonally appear bright while horizontal and vertical domains appear dark. The dark arrows indicate long axis of tubes. FIG. 3C: Panel a: The ˜0.5 mm tube section was probed using small angle X-ray scattering (SAXS). The sample was oriented with the long axis vertical. The SAXS pattern shows an obvious pinched profile indicating nanofiber alignment along the long axis of the tube. Panel b: An unaligned gel was also probed to show contrast between aligned and non-aligned SAXS data.

FIG. 4. Schwann cell proliferation in various gels. IKVAV-PA (SEQ ID NO: 2) statistically increased Schwann cell proliferation vs. backbone-PA up to Day 14; RGDS did so up to day 21. All p values were calculated using ANOVA and TukeyeKramer test. *p<0.05 vs. backbone-PA at day 1. Xp<0.05 vs. backbone-PA at day 7. zp<0.05 vs. backbone-PA at day 14. ¶p<0.05 vs. backbone-PA at day 21.

FIG. 5. Immunocytochemistry of actin and focal adhesion protein (FAP) of Schwann cells on conventional 2D culture dish (2D), in collagen gel, aligned backbone-PA gel, aligned IKVAV-PA (SEQ ID NO: 2) gel, or aligned RGDS-PA (SEQ ID NO: 1) gel. The white arrows indicate the direction of the gel. Scale bar, 50 mm.

FIG. 6. Gross observation of sciatic nerve 12 weeks after the surgery. (Panel A) No nerve regeneration was observed in the negative control; scar tissue filled the original defect site. Arrow indicates the nerve stump. (Panel B) Nerve bridging was observed in the Autograft group. Arrows indicate the original proximal and distal nerve stumps. (Panel C) Nerve regeneration was observed in the PLGA/RGDS-PA (SEQ ID NO: 1) group. PLGA conduit was degraded completely, leaving behind regenerate nerve structure. Arrows indicate the original proximal and distal nerve stumps.

FIG. 7. Hind paw latency to withdrawal in seconds plotted against postoperative day. Results from the various experimental conditions are presented.

FIG. 8. Gait was measured by examination of footprint patterns during ambulation at different time points (4, 8, and 12 weeks) following sciatic nerve transection. This data was used to calculate the SFI. *p<0.05 vs. empty conduit at Week 4.

FIGS. 9A-D. Histological findings at postoperative week 12. FIG. 9A: The transverse sections of autograft, empty conduit, backbone-PA, and PLGA/RGDS-PA(SEQ ID NO: 1) groups compared with that of normal sciatic nerve were analyzed by H.E. staining. FIG. 9B: Immunohistochemistry staining with anti-neurofilament antibody (NF) in transverse sections of autograft, empty conduit, backbone-PA, and PLGA/RGDS-PA(SEQ ID NO: 1) groups compared with that of normal control nerves. The nuclei were counterstained with hematoxylin. FIG. 9C: Immunohistochemistry staining of the Schwann cell marker S-100 in transverse sections of autograft, empty conduit, backbone-PA, and PLGA/RGDS-PA(SEQ ID NO: 1) groups compared with that of normal nerve. The nuclei were counterstained with hematoxylin. Bar ¼ 50 mm FIG. 9D: The number of NF (left) and S-100 (right) positive cells of each groups. *p<0.05 vs. empty conduit.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, the term “peptoid” refers to a class of peptidomimetics where the side chains are functionalized on the nitrogen atom of the peptide backbone rather than to the α-carbon.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence “having at least Y % sequence identity with SEQ ID NO:Z” may have up to X substitutions relative to SEQ ID NO:Z, and may therefore also be expressed as “having X or fewer substitutions relative to SEQ ID NO:Z.”

As used herein, the term “nanofiber” refers to an elongated or threadlike filament (e.g., having a significantly greater length dimension that width or diameter) with a diameter typically less than 100 nanometers.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, marcomolecules, etc.) and the multicomponent assemblies, complexes, systems, and/or fibers that form as a result.

As used herein, the term “physiological conditions” refers to the range of conditions of temperature, pH and tonicity (or osmolality) normally encountered within tissues in the body of a living human.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide lipophilic (hydrophobic) segment, a structural peptide segment and/or charged peptide segment (often both), and optionally a bioactive segment (e.g., linker segment, bioactive segment, etc.). The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment (e.g., β-sheet forming); (3) a charged peptide segment, and (4) a bioactive segment (e.g., linker segment).

As used herein and in the appended claims, the term “lipophilic moiety” or “hydrophobic moiety” refers to the moiety (e.g., an acyl, ether, sulfonamide, or phosphodiestermoiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)— where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used herein, the term “structural peptide” refers to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural segments of adjacent structural segments. In some embodiments, nanofibers of peptide amphiphiles having structural peptide segments display linear or 2D structure when examined by microscopy and/or α-helix and/or β-sheet character when examined by circular dichroism (CD).

As used herein, the term “beta (β)-sheet-forming peptide segment” refers to a structural peptide segment that has a propensity to display β-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises one or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, and or about 3 or 4 amino acids in this segment.

As used herein, the terms “amino-rich peptide segment”, “basic peptide segment,” and “positively-charged peptide segment” refer to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A basic peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., IKVAV(SEQ ID NO: 2), RGDS(SEQ ID NO: 1), etc.) exhibits the functionality of the bioactive peptide.

### DETAILED DESCRIPTION

Peptide amphiphiles (PAs) are molecules that self-assemble into nanofibers, which can be aligned by physical manipulation (e.g., to mimic the native architecture of peripheral nerve). Experiments were conducted during development of embodiments of the present invention to demonstrate that PAs are a useful substrate for use as a bioengineered nerve graft substitute. To examine this, Schwann cells were cultured with bioactive PAs (e.g., RGDS-PA(SEQ ID NO: 1) (See e.g., U.S. Pat. No. 7,491,690, U.S. Pat. No. 8,076,295, and U.S. Pat. No. 8,834,840, herein incorporated by reference in their entireties), IKVAV-PA(SEQ ID NO: 2) (See, e.g., U.S. Pat. No. 8,748,569, U.S. Pat. No. 8,076,295, U.S. Pat. No. 8,834,840, and U.S. Pat. No. 8,063,014, herein incorporated by reference in their entireties) to determine their ability to attach to and proliferate within the biomaterial. Next, a PA construct was devised for use in a peripheral nerve critical sized defect model. Rat sciatic nerve defects were created and reconstructed with autologous nerve, PLGA conduits filled with various forms of aligned PAs, or left unrepaired. Motor and sensory recovery were determined and compared among groups. Results demonstrate that Schwann cells are able to adhere to and proliferate in aligned PA gels, with greater efficacy in bioactive PAs compared to the backbone-PA alone. In vivo testing revealed recovery of motor and sensory function in animals treated with conduit/PA constructs comparable to animals treated with autologous nerve grafts. Bioactive recovery in conduit/PA and autologous graft groups was significantly faster than in animals treated with empty PLGA conduits. Histological examinations also demonstrated increased axonal and Schwann cell regeneration within the reconstructed nerve gap in animals treated with conduit/PA constructs. These results demonstrate that PA nanofibers are a useful biomaterial for use in, for example, bioengineered peripheral nerve repair.

Experiments were conducted during development of embodiments of the present invention to demonstrate the use of aligned nanofiber gels formed by self-assembling peptide amphiphiles (PA) in peripheral nerve regeneration (refs. 10,11; herein incorporated by reference in their entireties). Because of their unique properties and molecular design, PA nanofibers mimic the internal fascicular architecture of peripheral nerves, allow for the incorporation of Schwann cells vital for peripheral nerve regeneration, induce cellular and neurite alignment, and guide cell migration (ref. 12; herein incorporated by reference in its entirety). Moreover, in some embodiments, PAs are engineered to possess bioactivity that is relevant to nerve regeneration. A PA presenting the amino acid sequence IKVAV (SEQ ID NO: 2) (derived from laminin) has been shown to induce neural stem cell differentiation, stimulate neurite outgrowth, and lead to bioactive improvement in acute spinal-cord injury (refs. 13,14; herein incorporated by reference in its entirety). The amino acid sequence RGDS (SEQ ID NO: 1) is found on molecules of fibronectin (and other proteins) and PAs bearing this sequence have been shown to promote cellular motility, proliferation, and differentiation in vitro (ref. 15-21; herein incorporated by reference in their entireties).

The natural anatomy of a peripheral nerve fiber provides the basis for its regeneration in the setting of injury. The outer epineurial layer maintains nerve fiber integrity and assists with its incorporation into the surrounding soft tissues, while the internal fascicular structure supports axonal guidance, function and proliferation. On the molecular level, bioactive extracellular matrix (ECM) molecules, in particular laminin and fibronectin, play a crucial role in stimulating various cellular activities that are integral to regeneration and facilitating Schwann cell migration, and proliferation, which is critical for neurite outgrowth (ref. 26; herein incorporated by reference in its entirety). These molecules also provide binding sites and directional guidance for growing axons.

Much research on peripheral nerve regeneration has focused on the development of nerve conduits for guided nerve regeneration. Axons sprout from the proximal end of the transected nerve end and grow into the conduit, which bridges the nerve gap. The conduit serves to guide and protect the nerve regenerate as it approaches the distal end of the cut nerve. Currently, PLGA-based and collagen-based conduits are commercially available for use in peripheral nerve repair in humans. However, the efficacy of these empty conduits is limited to short distance nerve gaps, on the order of 1 cm to less than 3 cm (ref. 8; herein incorporated by reference in its entirety). Beyond that length, the efficacy of nerve repair using a conduit declines with increasing nerve gap size. Acellular cadaveric nerve allografts have been reported to be effective in defects up to at least 5 cm in length (ref. 9; herein incorporated by reference in its entirety).

PA nanofibers present a biomaterials platform for addressing the requirements that need to be met in the design of a nerve graft substitute. PA molecules comprise 4 bioactive segments: 1) A hydrophobic alkyl tail (e.g. palmitoyl) that drives aggregation through hydrophobic collapse; 2) a beta-sheet-forming peptide sequence (e.g. VVAA (SEQ ID NO: 4)) that promotes nanofiber formation and influences the fiber mechanical properties; 3) ionizable side-chain residues that render PAs soluble in aqueous solutions (e.g. E); and 4) a bioactive epitope sequence (e.g., bioactive sequence) that can interact with cellular receptors and stimulate cellular activities. Upon thermal treatment, certain PA sequences (e.g. VVAAEE (SEQ ID NO: 3)) form liquid crystal-like fiber bundles that can be aligned upon application of a gentle shear (similar to that of pipetting). Aligned PAs are able to gel in the presence of salts with divalent cations (such as Ca2 [symbol] ), and the forces required for alignment are much lower than with the high strain or flow rates required in electrospun internal scaffolds (ref. 10; herein incorporated by reference in its entirety). This physiologic fabrication process allows living cells to be cultured directly into the biomaterial as it undergoes gelation.

The benefits of the IKVAV (Ile-Lys-Val-Ala-Val) (SEQ ID NO: 2) epitope have already been shown in spinal cord injury models (ref. 13,14; herein incorporated by reference in their entireties). As an integrin binding sequence found on fibronectin, RGDS (Arg-Gly-Asp-Ser) (SEQ ID NO: 1) exerts key biochemical influences on a variety of cells types including fibroblasts (ref. 15; herein incorporated by reference in its entirety), osteoblasts (refs. 34,35; herein incorporated by reference in their entireties), and neurons (ref. 36; herein incorporated by reference in its entirety). Schwann cells are also influenced by interactions with fibronectin, and these cells are vital to axonal growth and elongation by creating the specific biochemical microenvironment needed for peripheral nerve regeneration. In particular, Schwann cells phagocytose axonal and myelin debris and elaborate chemo attractant molecules for macrophages that help in the same manner. The naturally occurring RGDS (SEQ ID NO: 1) sequences found in fibronectin play a vital role in the stimulation and regulation of Schwann cell activity in the regeneration of peripheral nerve.

Experiments conducted during development of embodiments of the present invention have demonstrated that Schwann cells remain viable in aligned PA gels, and furthermore that Schwann cell proliferation in these gels is comparable to that seen in collagen conduit, including after 21 days of culture (FIG. 4). This result indicates that PAs may be used clinically in a bioengineered nerve graft construct without negative effects on the growth kinetics of the vital cellular components needed for peripheral nerve regeneration.

Experiments conducted during development of embodiments of the present invention have also shown that the addition of the use of bioactive PAs significantly increases the proliferation of Schwann cells (FIG. 4). Both IKVAV-PA (SEQ ID NO: 2) and RGDS-PA (SEQ ID NO: 1) increased proliferation of Schwann cells compared to backbone-PA for at least 14 days. This observation was persistent through 21 days in the RGDS-PA (SEQ ID NO: 1) model, and is consistent with the fibronectin-like conformation of the RGDS (SEQ ID NO: 1) epitope and its role as discussed above. Based on this finding, RGDS-PA (SEQ ID NO: 1) was used in in vivo nerve regeneration experimental groups.

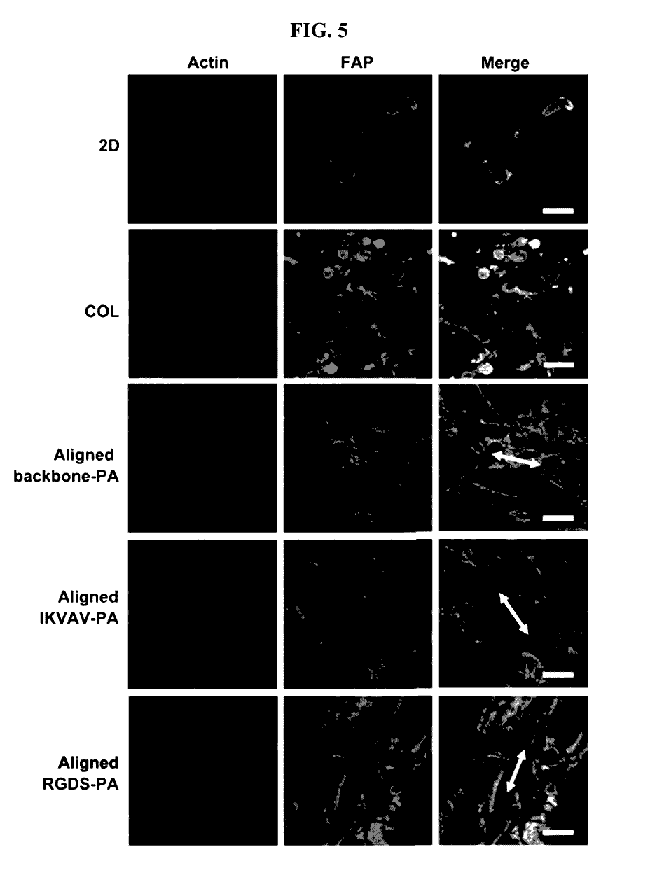

While proliferation assays provide an idea of the health of the Schwann cells in three-dimensional in vitro culture, it is useful to document that these cells are behaving in a manner that is consistent with their behavior in native nerve tissue. Although proliferation assays demonstrate increasing proliferation, they do not indicate if the resulting growth is organized in an aligned and guided fashion that is conducive to effective peripheral nerve regeneration. Through the use of immunocytochemistry (ICC), however, it was demonstrated that Schwann cells orient along the aligned axis of PA gels (FIG. 5). While Schwann cells were able to grow and attach to collagen matrices in control cultures, they did so in an unaligned fashion: cytoskeletal elements and process extensions were randomly oriented. In contrast, cells grown in aligned PA gels—both backbone-PA and bioactive PAs—demonstrated marked linear arrangement of their cytoskeletal elements, their pattern of growth, and their cellular extensions.

In vivo results further demonstrate the value of providing an internal scaffolding to the nerve regeneration construct. The rat sciatic nerve model is a widely recognized and implemented in the study of peripheral nerve injury (ref. 37; herein incorporated by reference in its entirety). While 10 mm defects have been used previously by other groups (ref. 38; herein incorporated by reference in its entirety), a larger defect was used in experiments conducted during development of embodiments of the present invention to mitigate any chance of spontaneous autologous repair. As negative controls demonstrate, there was no return of sensory or motor function in animals with defects that were left unrepaired. Furthermore, histological examination of the surgical sites in negative control animals revealed no regeneration of nerve tissue within the critical defects 12 weeks after surgery.

The SFI is frequently used in the assessment of peripheral nerve motor function as modeled using the rat sciatic nerve (refs. 39,40; incorporated by reference in their entireties) and the use of thermal sensitivity/nocioception is also a commonly used assessment tool (refs. 41,42; incorporated by reference in their entireties). The bioengineered PLGA/RGDS-PA (SEQ ID NO: 1) constructs yielded return of motor function that was not only statistically improved from empty conduit, but that was also statistically comparable to results observed in animals treated with autologous nerve graft, the current gold standard in nerve repair therapy. There was a trend of improvement when comparing empty conduit to PLGA/backbone-PA construct, but these differences were not statistically significant. Overall, the PLGA/RGDS-PA (SEQ ID NO: 1) construct demonstrated a quicker return of motor function in experimental animals. Based on in vitro data, this is attributed in part to its ability to augment Schwann cell alignment, proliferation, and migration through its fibronectin-mimicking characteristics. If these vital regenerative cells are able to grow more quickly within the defect site, the optimal biochemical environment needed for nerve regeneration may develop more efficiently.

Similar results were noted in sensory function assessments. Thermal nocioceptive signals are conveyed via unmyelinated C fibers, which are responsible for the delayed sensation associated with heat-associated pain (refs. 43,44; herein incorporated by reference in their entireties). Latency time from introduction of a thermal noxious stimulus (i.e., a burning sensation) to actual withdrawal of the hind paw from the heat source was used as a measure of intact sensory function. In unoperated control limbs, this time averaged 5 s (range 4e6 s). Interestingly, both the PLGA/backbone-PA and PLGA/RGDS-PA (SEQ ID NO: 1) constructs demonstrated significantly faster return of sensory function compared to empty conduit, approaching test results seen in normal limbs after 30 days. Rate of recovery was also comparable to that seen in autologous reconstruction groups, and in fact was improved during the first week following surgery. Unreconstructed groups never saw return to normal latency times. These results further underscore the positive impact of an internally aligned architecture in the design of the nerve regeneration construct, again emphasizing the potential role of the bioactive PA epitope in supporting Schwann cell activity and subsequent bioactive outcomes.

Histological analysis of the reconstructed defect sites is consistent with in vivo bioactive test findings. Both axons and Schwann cells were able migrate into and proliferate within the PLGA/backbone-PA and PLGA/RGDS-PA (SEQ ID NO: 1) constructs. Notably, empty conduits demonstrated negligible axonal and Schwann cell migration. The architecture of the regenerate nerves in autologous and PA-based reconstructions was reminiscent of the native nerve architecture seen in histological specimens of unoperated nerve controls.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, embodiments described herein encompasses peptide amphiphiles having a C-terminal moiety that may be selected from the group consisting of —H, —OH, —COOH, —CONH2, and —NH2.

In some embodiments, peptide amphiphiles comprise a hydrophobic (non-peptide) segment linked to a peptide. In some embodiments, the peptide comprises a structural segment (e.g., hydrogen-bond-forming segment, beta-sheet-forming segment, etc.), and a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more bioactive groups (e.g., alkene, alkyne, azide, thiol, etc.) for the attachment of a mono-, di-, oligo-, or polysaccharide, or glycomimetic residue. In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N-terminus of the peptide after the last amino acid coupling, and is composed of a fatty acid or other acid that is linked to the N-terminal amino acid through an acyl bond. In aqueous solutions, PA molecules self-assemble (e.g., into cylindrical micelles (a.k.a nanofibers)) that bury the lipophilic segment in their core and display the bioactive peptide on the surface. The structural peptide undergoes intermolecular hydrogen bonding to form beta sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment (e.g., a peptide comprising a segment having a preference for beta-strand conformations) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture. In various embodiments, hydrophobic segments pack in the center of the assembly with the peptide/saccharide segments exposed to an aqueous or hydrophilic environment to form cylindrical nanostructures that resemble filaments. Such nanofilaments display the peptide or saccharide regions on their exterior and have a hydrophobic core.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segment may comprise natural amino acids, modified amino acids, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises EE.

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K.

In some embodiments, peptide amphiphiles comprises a structural and/or beta-sheet-forming segment. In some embodiments, the structural segment is rich in H, I, L, F, V, and A residues. In some embodiments, the structural and/or beta-sheet-forming segment comprises an alanine- and valine-rich peptide segment (e.g., AAVV(SEQ ID NO: 5), AAAVVV(SEQ ID NO: 6), or other combinations of V and A residues, etc.). In some embodiments, the structural and/or beta sheet peptide comprises 4 or more consecutive A and/or V residues, or conservative or semi-conservative substitutions thereto. In some embodiments, the structural and/or beta-sheet forming peptide segment comprises 4 or more consecutive non-polar aliphatic residues (e.g., alanine (A), valine (V), leucine (L), isoleucine (I), methionine (M)). In some embodiments, the structural and/or beta-sheet forming peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group. In some embodiments, the spacer or linker segment provides a reactive group (e.g., alkene, alkyne, azide, thiol, maleimide etc.) for functionalization (e.g., glycosylation) of the PA. In some embodiments, the spacer or linker is a substantially linear chain of CH2, O, (CH₂)₂O, O(CH₂)₂, NH, and C═O groups (e.g., CH2(O(CH₂)₂)₂NH, CH2(O(CH₂)₂)₂NHCO(CH₂)₂CCH, etc.). In some embodiments, a spacer or linker further comprises additional bioactive groups, substituents, branches, etc.

Suitable peptide amphiphiles, PA segments, PA nanostructures, and associated reagents and methods are described, for example in U.S. Pat. No. 8,512,693; U.S. Pat. No. 8,450,271; U.S. Pat. No. 8,138,140; U.S. Pat. No. 8,124,583; U.S. Pat. No. 8,114,835; U.S. Pat. No. 8,114,834; U.S. Pat. No. 8,080,262; U.S. Pat. No. 8,063,014; U.S. Pat. No. 7,851,445; U.S. Pat. No. 7,838,491; U.S. Pat. No. 7,745,708; U.S. Pat. No. 7,683,025; U.S. Pat. No. 7,554,021; U.S. Pat. No. 7,544,661; U.S. Pat. No. 7,534,761; U.S. Pat. No. 7,491,690; U.S. Pat. No. 7,452,679; U.S. Pat. No. 7,390,526; U.S. Pat. No. 7,371,719; U.S. Pat. No. 6,890,654; herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural segment (e.g., comprising VVAA(SEQ ID NO: 4)); and (c) a charged segment (e.g., comprising EE). In some embodiments, the peptide amphiphile further comprises an attachment segment (e.g., K) for attachment of a bioactive group (e.g., attachment of a spacer, glycosylation, etc.). In some embodiments, any PAs within the scope described herein, comprising the components described herein, or within the skill of one in the field, may find use herein.

In some embodiments, peptide amphiphiles comprise a bioactive moiety. In particular embodiments, a bioactive moiety is the C-terminal most segment of the PA. In some embodiments, the bioactive moiety is attached to the C-terminal end of the charged segment. In some embodiments, the bioactive moiety is exposed on the surface of a assembled PA structure (e.g., nanofiber). A bioactive moiety is typically a peptide (e.g., IKVAV (SEQ ID NO: 2), RGDS (SEQ ID NO: 1), etc.), but is not limited thereto. Examples described in detail herein utilize a peptide sequence that binds IKVAV (SEQ ID NO: 2) and RGDS (SEQ ID NO: 1) as a bioactive moiety. Bioactive peptides and other moieties for achieving functionality will be understood. In some embodiments, bioactive moieties are provided having binding affinity for a target protein. The binding affinity (K_{d}) may be chosen from one of: less than 10 μM, less than 1 μM, less than 100 nM, less than 10 nM, less than 1 nM, less than 100 μM.

Suitable peptide amphiphiles, PA segments, PA nanostrcutures, and associated reagents and methods for use in some embodiments herein are described, for example in U.S. Pat. No. 8,512,693; U.S. Pat. No. 8,450,271; U.S. Pat. No. 8,138,140; U.S. Pat. No. 8,124,583; U.S. Pat. No. 8,114,835; U.S. Pat. No. 8,114,834; U.S. Pat. No. 8,080,262; U.S. Pat. No. 8,063,014; U.S. Pat. No. 7,851,445; U.S. Pat. No. 7,838,491; U.S. Pat. No. 7,745,708; U.S. Pat. No. 7,683,025; U.S. Pat. No. 7,554,021; U.S. Pat. No. 7,544,661; U.S. Pat. No. 7,534,761; U.S. Pat. No. 7,491,690; U.S. Pat. No. 7,452,679; U.S. Pat. No. 7,390,526; U.S. Pat. No. 7,371,719; U.S. Pat. No. 6,890,654; herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural segment, bioactive segment, etc.). For example, nanofibers, nanospheres, intermediate shapes, and other supramolcular structures are achieved by adjusting the identity of the PA component parts.

In some embodiments, nanostructures (e.g., nanofibers) assembled from the peptide amphiphiles described herein are provided. In some embodiments, nanostructures are assembled from PAs bearing a bioactive moiety. In some embodiments, nanostructures are assembled from (1) PAs bearing a bioactive moiety and (2) filler PAs (e.g., PAs not-labeled or not displaying a bioactive moiety, etc.). In some embodiments, nanostructures (e.g., nanofibers) comprise: (i) less than 50% (e.g., 49%, 45%, 40%, 35%, 30%, 25%, 20%, 15%, 10%, 5%, 4%, 3%, 2%, 1%, or any ranges there between) PAs bearing a bioactive moiety. In some embodiments, nanostructures (e.g., nanofibers) comprise and at least 2% (e.g., 2%, 3%, 4%, 5%, 10%, 15%, 20%, 25%, 30%, 35%, 40%, 45%, 50%, 55%, 60%, 65%, 70%, 75%, 80%, 85%, 90%, 95%, 98%, or any ranges there between) PAs bearing a bioactive moiety. In some embodiments, nanofibers comprise at least 50% (e.g., 50%, 55%, 60%, 65%, 70%, 75%, 80%, 85%, 90%, 95%, 98%, or any ranges there between) filler peptide amphiphiles. In some embodiments, the ratio of PAs bearing a bioactive moiety to filler PAs determines the density of bioactive moieties displayed on the nanostructure surface.

In some embodiments, provided herein are biological scaffolds comprising a polymer conduit and the PAs described herein. In some embodiments, the conduit is a narrow (e.g., internal diameter of 0.1-4 mm (e.g., 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, or ranges therebetween), external diameter of 1-6 mm (e.g., 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm 5.1 mm, 5.2 mm, 5.3 mm, 5.4 mm, 5.5 mm, 5.6 mm, 5.7 mm, 5.8 mm, 5.9 mm, 6 mm, or ranges therebetween)). In some embodiments, the conduit is 5-100 mm (e.g., 5, 6, 7, 8, 9, 1−, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or ranges therebetween) in length.

In some embodiments, the conduit is composed of one or more materials which are biodegradable and/or biorespobable. In some embodiments, the conduit comprises one or more polymers. Suitable polymers include, for example, a polymer from the linear polyester family, such as polylactic acid, polyglycolic acid or polycaprolactone and their associated copolymers, e.g. poly(lactide-co-glycolide) at all lactide to glycolide ratios, and both L-lactide or D,L lactide. Polymers such as polyorthoester, polyanhydride, polydioxanone and polyhyroxybutyrate may also be employed. In some embodiments, a conduit comprises PLGA. In other embodiments, the conduit comprises an aliphatic polyester, a polyanhydride, a polyphosphazine, a polyvinyl alcohol, a polypeptide, an alginate, or any combination thereof.

Conduits used in embodiments herein may comprise any suitable materials (e.g., polymers) that are useful in chemical and/or biochemical synthesis. Such materials may include glasses, silicates, celluloses, synthetic resins, and polymers. Suitable polymers may include those listed in the preceding paragraph as well as others including, but not limited to: substantially pure carbon lattices (e.g., graphite), dextran, polysaccharides, polypeptides, polynucleotides, acrylate gels, polyanhydride, poly(lactide-co-glycolide), polytetraflouroethylene, polyhydroxyalkonates, cross-linked alginates, gelatin, collagen, cross-linked collagen, collagen derivatives, such as, succinylated collagen or methylated collagen, cross-linked hyaluronic acid, chitosan, chitosan derivatives, such as, methylpyrrolidone-chitosan, cellulose and cellulose derivatives such as cellulose acetate or carboxymethyl cellulose, dextran derivatives such carboxymethyl dextran, starch and derivatives of starch such as hydroxyethyl starch, other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homoploymers, polyoxanones and polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin, silicone rubbers, or polyurethanes, and biocompatible and/or biodegradable derivatives and/or combinations thereof.

In particular embodiments, the compositions and methods herein find use in nerve regeneration; however, applications are not so limited. Compositions and methods herein may find use more broadly in other tissue regeneration applications, other medical applications, or other non-medical materials applications. In some embodiments, the PAs, PA nanofibers, and/or conduit/PA constructs described herein are useful in a range of applications, in vitro and/or in vivo, for example, stimulation of cell or tissue growth and/or proliferation.

In some embodiments, a method of preventing or treating injury or disease, tissue regeneration, etc. in a subject in need of such treatment is provided, the method comprising administering an effective amount of the PAs, nanostructures self-assembled therefrom, or conduit constructs comprising as much to the subject. The administered PAs, nanostructures, and/or conduit/PA constructs may be formulated in a suitable pharmaceutical composition or medicament and may further comprise a pharmaceutically acceptable carrier, adjuvant or diluent.

In some embodiments, methods are useful for promoting nerve repair (e.g., peripheral nerve repair, for example, by administering compositions herein to an injured or diseased nerve.

In some embodiments, methods are useful for promoting the formation of soft tissues (e.g., tendons, ligaments, fascia, skin, fibrous tissues, fat, synovial membranes, muscles, nerves, blood vessels, etc.), for example, by administering PAs described herein, nanostructures self-assembled therefrom, and/or conduit/PA constructs thereof to precursor cells for the desired soft tissue.

In some embodiments, methods are useful for promoting the formation of tissues (e.g., soft tissues, nerve tissue, etc.). The prevention or treatment of disease using the PAs and supramolecular nanostructures herein may involve the repair, regeneration or replacement of tissue, particularly soft or nerve tissue such. In subjects having a deterioration of one of these tissues (e.g., due to disease or injury), administration of PAs described herein, nanostructures self-assembled therefrom, and/or conduit/PA constructs thereof to the site of deterioration may be used to stimulate the growth, proliferation and/or differentiation of tissue at that site.

Alternatively, cells or tissue obtained from culture (e.g., of nerve cells, of nerve precursor cells, etc.) in contact with PAs described herein, nanostructures self-assembled therefrom, and/or conduit/PA constructs thereof is collected and implanted at the site of injury or disease to replace damaged or deteriorated tissue. The damaged or deteriorated tissue may optionally first be excised from the site of injury or disease. A method of implantation of cells and/or tissues is provided comprising the steps of: (a) culturing cells and/or tissues (e.g., nerve cells or tissue) in vitro in contact with the PAs described herein, nanostructures self-assembled therefrom, and/or conduit/PA constructs thereof; (b) collecting the cells and/or tissues; and (c) implanting the cells and/or tissues into a human or animal subject in need of treatment (e.g., at a site in need of tissue regeneration). In some embodiments, the cells/tissues are cultured in part in contact with PAs described herein, nanostructures self-assembled therefrom, and/or conduit/PA constructs thereof for a period of time sufficient to allow growth, proliferation or differentiation of the cells or tissues. For example, the period of time may be chosen from: at least 5 days, at least 10 days, at least 20 days, at least 30 days or at least 40 days.

In some embodiments, a composition is provided containing cells (e.g., nerve cells, nerve precursor cells), and PAs described herein, nanostructures self-assembled therefrom, and/or conduit/PA constructs thereof. Administration, e.g. injection, of the composition at the site of injury, disease or deterioration provides for the regeneration of tissue (e.g., nerve tissue) at the site.

In some embodiments, a pharmaceutical or medical composition or medicament comprising PAs described herein, nanostructures self-assembled therefrom, and/or conduit/PA constructs thereof is provided, optionally in combination with a pharmaceutically acceptable carrier, adjuvant or diluent. In some embodiments pharmaceutical compositions or medicaments may further comprise other agents useful in regenerative medicine. Pharmaceutical compositions or medicaments comprising are provided for use in, for example, the prevention or treatment of injury or disease, tissue regeneration, etc. The use of PAs described herein, nanostructures self-assembled therefrom, and/or conduit/PA constructs thereof in the manufacture of a medicament for the prevention or treatment of injury or disease, tissue regeneration, etc. is also provided.

### EXPERIMENTAL

### Example 1

### Materials and Methods

### Rat Schwann Cells Cultures

Cells were purchased from ATCC (cell line RT4-D6P2T) and cultured in Dulbecco's Modified Eagle's Medium (DMEM) with 10% fetal bovine serum (FBS). Cells were cultured to confluence, treated with 0.25% trypsin, centrifuged, and resuspended in PBS for a final concentration of 3500 cells/ml for WST-1 proliferation assays and 10,000 cells/ml for immunocytochemistry (ICC).

### Backbone-PA and Bioactive PA Gels

In order to synthesize the backbone-PA (consisting of only palmitoyl-VVAAEENH₂) (SEQ ID NO: 3), the molecule was dissolved in aqueous 150 mM NaCl, 3 mM KCl, and NaOH solution to obtain a final 1 wt % solution at a pH between 7.2 and 7.4. In order to fabricate mixtures containing epitope-bearing PAs IKVAV (SEQ ID NO: 2) and RGDS (SEQ ID NO: 1) (consisting of palmitoyl-VVAAEE-NH₂(SEQ ID NO: 3) with appended peptides, either RGDS (Arg-Gly-Asp-Ser) (SEQ ID NO: 1) or IKVAV (Ile-Lys-Val-Ala-Val) (SEQ ID NO: 2)), the backbone-PA was dissolved in aqueous 150 mM NaCl, 3 mM KCl, and NaOH solution to obtain a 1.33 wt % solution at a pH between 7.2 and 7.4. The epitope-containing PA was then dissolved with the same solution to obtain a 1 wt % solution at a pH between 7.2 and 7.4. The 1.33 wt % PA solution was then mixed in a 3:1 ratio with the 1% bioactive epitope solution creating a final bioactive PA solution of 1 wt % PA and 0.25 wt % bioactive epitope. The three resulting solutions (backbone-PA, RGDS-PA (SEQ ID NO: 1), and IKVAV-PA (SEQ ID NO: 2)) were then heated to 80° C. for 30 min in a water bath and left in the bath for slowcooling to 37° C. overnight (refs. 10,11; herein incorporated by reference in their entireties).

Combining Backbone-PA and Bioactive PA with Schwann Cells

Following completion of the slow cooling, each of the PA solutions was mixed with Schwann cell suspension in a 4:1 ratio. A final cell density of 3500 cells/ml was used for the WST-1 proliferation assays and 10,000 cells/ml for immunocytochemistry studies.

### Backbone-PA and Bioactive PA-Cell Gel Fabrication

A 200 ml volume of gelling solution consisting of 20 mM CaCl₂, 150 mM NaCl, and 3 mM KCl was pipetted onto a glass slide to form a thin film. The PA-Schwann cell suspensions were then pipetted in 4 ml aliquots onto the thin film, instantly forming a string-like gel, with cells encapsulated within. These cell-embedded gels were maintained free floating in DMEM with 10% FBS in culture plates and incubated at 37° C. Medium was changed every 72 h.

### Collagen Gels

Volumetric ratios of 1 ml HEPES, 10 ml 10XPBS, 87 ml Collagen 10 mg/mL, and 2 ml Schwann cell PBS suspension were combined and mixed gently. Final cell density of 3500 cells/ml was used for the WST-1 proliferation assays and 10,000 cells/ml, for immunocytochemistry studies. The mixture was then pipetted into a culture well (either a 96-well plate or 2-welled microscopy slides), and after 1 h of curing and solidification at 37° C., medium was added gently to the wells and changed every 3 days.

### WST-1 Proliferation Assay

To evaluate the viability of cells within the gels, WST-1 reagent (Roche Applied Science, Indianapolis, Ind., USA) was used for each PA-cell and collagen gelecell complex. WST-1 assay has been widely used to measure cell proliferation. Aligned PA-cell gels along with collagen-cell gels were fabricated through the aforementioned methods at a final cell density of 3500 cells/ml. Gels were then cultured in 96-well plates for various time periods (days 1, 7, 14, and 21). Following each time point, gels were incubated for 90 min in medium containing 10% WST-1 assay reagent. Following incubation, 100 mL of the conditioned medium were then transferred to a 96-well reading plate, and absorption values were read in a spectrophotometer at 450 nm. Measurements were repeated in sets of four.

### Immunocytochemistry

PA-cell gels along with collagen-cell gels were fabricated through the aforementioned methods at a final cell density of 10,000 cells/ml. These gels were then cultured for 48 h in 2-well microscopy slides, followed by fixation with 10% formalin and staining with mouse anti-vinculin monoclonal antibody (Abcam, Cambridge, Mass.), followed by FITC-conjugated anti-mouse secondary antibody (Abcam). Actin filaments were stained with rhodamine phalloidin (Life Technologies, Carlsbad, Calif.). Specimens were then viewed with confocal microscopy (Confocal SP1 MP-Inverted, Leica Microsystems Inc., Buffalo Grove, Ill.) using oil magnification, and images were recorded.

### Preparation of PLGA Tube

Poly (lactide-co-glycolide acid) (PLGA) (75:25 mol ratio of D, L-lactide to glycolide, 0.76 dL/g, Lakeshore Biomaterials, Birmingham, Ala.) tubes were created using a modified gas foaming technique (ref. 22; herein incorporated by reference in its entirety). PLGA microspheres were prepared with dichloromethane in water emulsion technique. A 1:7 weight ratio mixture of PLGA microspheres and NaCl particles (38e63 mm) were loaded into the annular gap of two glass tubes having an outside diameter of 3 mm and inside diameter of 1.7 mm. The powder mixture was manually packed using another glass tube having the same dimension as the annular gap. The materials were then equilibrated with high pressure CO₂ gas (800 psi) for 16 h in a custom-made pressure vessel. Afterwards, the pressure was released over a period of 45 min, which fused adjacent microspheres creating a continuous polymer structure. The NaCl porogen was leached for 6 h in sterile milli-Q water. The tubes were then freeze dried overnight.

### Incorporation of Aligned PA Gel

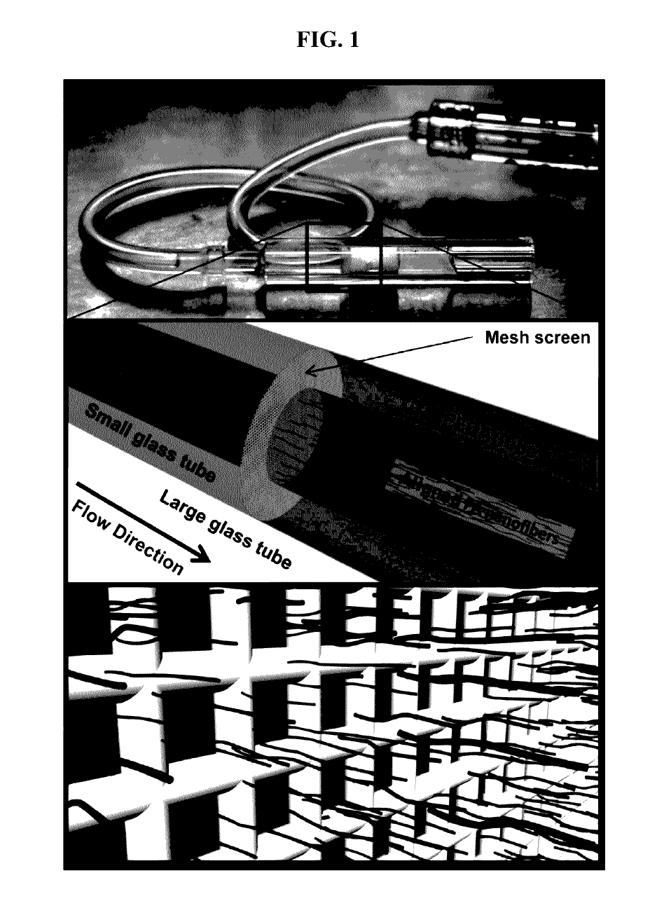

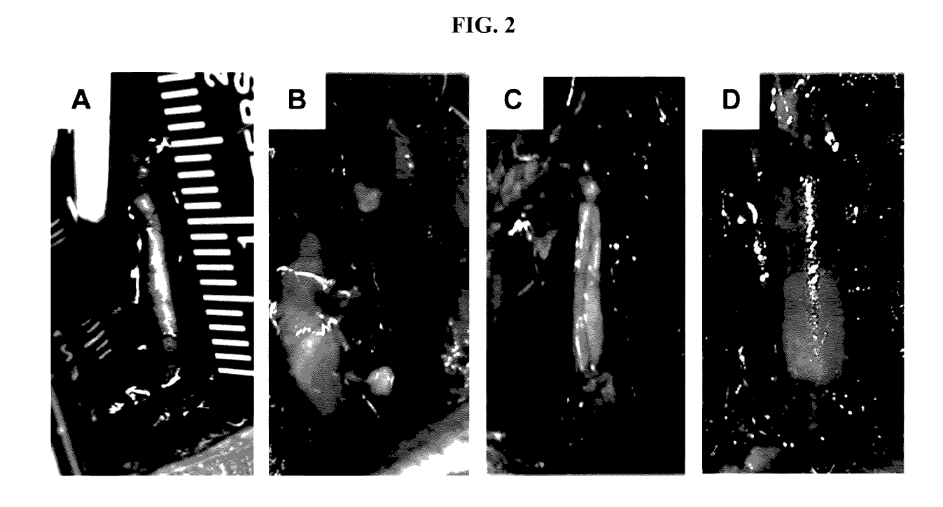

The porous PLGA tubes were then loaded with an aligned PA gel (FIG. 1). This was achieved by placing the PLGA tube inside a 3 mmID glass tube. Then, using a smaller 1.7 mmID glass tube, a 40 μm pore size mesh screen was fitted at the entrance of the PLGA tube. A syringe with a silicone tube fitting was used to force the PA nanofiber solution through the mesh screen, and inside the PLGA tube (FIG. 1). Annealed PA nanofiber solutions behave as liquid crystals having many microdomains of fiber alignment, which can be oriented unidirectionally through shear and elongational flow (ref. 10; herein incorporated by reference in its entirety). The liquid crystal-like PA solution experiences significant shear forces as it passes through the mesh screen, and the nanofibers respond by aligning in the direction of fluid flow. After the aligned PA nanofiber solution filled the PLGA tube, the entire glass tube containing the materials was submerged in a 20 mM CaCl₂ bath for 30 min allowing the PA solution to gel, thus trapping the nanofiber alignment in a gelatinous state. The PLGA/PA construct was then removed from the glass and cut to length, 12 mm, and stored at 4° C. for no longer than one week before implantation. Fabrication of backbone-PA and bioactive PA constructs followed the same protocol.

### SEM and Birefringence Imaging

In preparation for SEM imaging, PLGA/backbone-PA constructs were first cut in half lengthwise using a thin razor blade in order to expose the aligned backbone-PA gel within the PLGA shell. The sample was fixed in 3% glutaraldehyde for 20 min, and dehydrated in a series of EtOH washes. Samples were then critically point dried, mounted, and coated with 5 nm of osmium before imaging using a LEO Gemini 1525 sFEG SEM. The fiber alignment was also visualized using birefringence. Constructs were first embedded in an agarose gel block and manually sectioned into ˜0.5 mm slices cut parallel to the tube's long axis. These segments were laid flat in a glass dish filled with water and examined between two perpendicular light polarizers using an inverted Nikon Eclipse TPA00 Inverted Microscope.

### Sciatic Nerve Defect

Animal procedures and protocols were reviewed and approved by the Animal Research Committee of the University of California Los Angeles. Sprague Dawley rats (average weight: 250 g) were purchased from Charles River Laboratories (Wilmington, Mass., USA) and were housed and maintained at the UCLA vivarium under the care of the veterinary staff, according to the regulations set forth by the UCLA Office of Protection of Research Subjects. The rats were anesthetized using inhaled 2.5% isoflurane and the skin overlying the expected course of the sciatic nerve along the femur was trimmed of hair and prepped for surgery. An incision was made at the thigh and the sciatic nerve was carefully exposed and isolated within the intermuscular space. Soft tissues surrounding the nerve were dissected to alleviate any proximal or distal tension on the nerve trunk. Using a surgical microscope and microsurgical instruments, a 12 mm segment of the nerve was carefully measured and then excised. (FIG. 2A) The resultant defects were reconstructed according to the following conditions: 1) no repair (negative control): the defects were left unrepaired and the proximal and distal cut ends were sutured to surrounding adventitia to prevent nerve migration (FIG. 2B); 2) autograft (positive control): the resected nerve segment was immediately sutured back in situ (FIG. 2C); 3) empty conduit: a hollow 12 mm sleeve of PLGA was sutured to the proximal and distal ends of the defect; 4) PLGA/backbone-PA construct: a 12 mm segment of this construct was sutured to the proximal and distal ends of the defect; or 5) PLGA/RGDS-PA (SEQ ID NO: 1) construct: a 12 mm segment of this construct was sutured to the proximal and distal ends of the defect (FIG. 2D). The wound was then closed in layers, including muscle and skin. All the animals were kept under standardized laboratory conditions in an airconditioned room with free access to food and water.

### Sciatic Function Index

Motor bioactive recovery following the sciatic nerve injury was assessed using rat walking track analysis and footprint recording. The Sciatic Function Index (SFI) was calculated using the following formula:

SFI¼109:5 [symbol] ETS_NTS [symbol] =NTS_38:3 [symbol] EPL_NPL [symbol] =NPL [symbol] 13:3 [symbol] EIT_NIT [symbol] =NIT_8:8
(ref. 23; herein incorporated by reference in its entirety) where EPL indicates the operated experimental paw length; NPL, normal paw length; ETS, operated experimental toe spread, i.e., the distance between the first and fifth toes; NTS, the normal toe spread; EIT, the operated experimental intermediary toe spread, i.e., the distance between the second and fourth toes; and NIT, normal intermediary toe spread. Evaluations of all animals were performed by a single investigator blinded to the experimental repair conditions.

### Thermal Sensitivity Test

Sensory bioactive recovery following sciatic nerve injury was assessed using thermal sensitivity testing performed according to previously reported methods (ref. 24; herein incorporated by reference in its entirety). The Hargreaves paw withdrawal apparatus (Hargreaves Model 390, IITC Instruments) was used to measure the withdrawal latency from a radiant heat source directed at the proximal half of the plantar surface of each hind paw. Prior to testing, rats were allowed to acclimate for 10 min to the testing environment: a translucent plastic-walled individualized chamber (10×20×20 cm) with a 3 mm thick glass bottom that was preheated to 30° C. A radiant heat source consisting of an adjustable infrared lamp and a built-in stopwatch accurate to 0.1 s were used to measure paw withdrawal latency. Each paw was tested three times at 25% maximal heat intensity, with 5 min between each test. The test was performed only when a rat was stationary and standing on all four paws. Special care was taken to keep the glass bottom clean and dry during the testing. If the glass needed to be cleaned during the experiment, the rats were allowed 5-10 min to reacclimatize to the environment. The results of three tests were averaged for each paw.

### Histological Evaluations and Quantitative Analysis

The rats were euthanized using carbon dioxide followed by decapitation at week 12 following surgery. The sciatic nerve was re-exposed and harvested. The nerve specimens were fixed in 4% formaldehyde for 48 h, embedded in paraffin, and cut into 4 mm sections. These were then stained with conventional hematoxylin-eosin. Immunohistochemistry was also performed. Mouse monoclonal antibody to rat neurofilament (anti-NF, 1:1000 dilution; Abcam, Cambridge, Mass.) and S-100 (1:2000 dilution; SigmaeAldrich, St. Louis, Mo.) were applied overnight at 4° C., respectively. The signal was detected using the mouse DAKO horseradish peroxidase (Dako Corporation, Carpinteria, Calif.) and visualized with the diaminobenzidine reaction. The sections were counterstained with hematoxylin. The numbers of NF or S-100 positive cells were counted per square area (100×100 mm) using ImageJ software (National Institute of Health, Bethesda, Md.). Quantitative analyses of slides representing the various experimental conditions were compared.

### Statistical Analysis

A one-way analysis of variance (ANOVA) was used to compare groups. The TukeyeKramer multi-comparison adjustment was used as the post-hoc test to calculate the significance levels. p<0.05 was considered statistically significant.

### Example 2

### Results

### Morphology of Aligned PA Gel

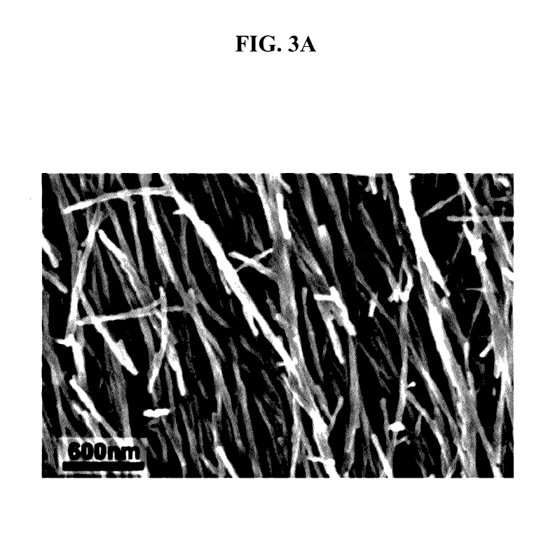

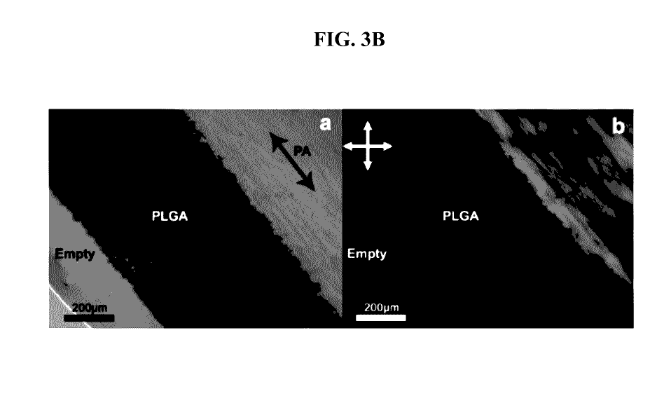

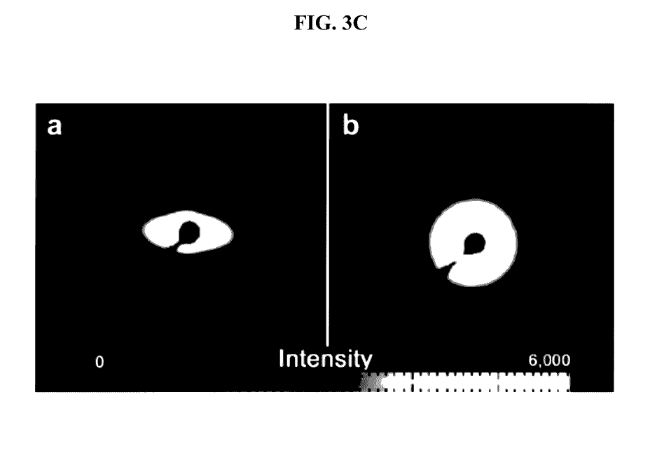

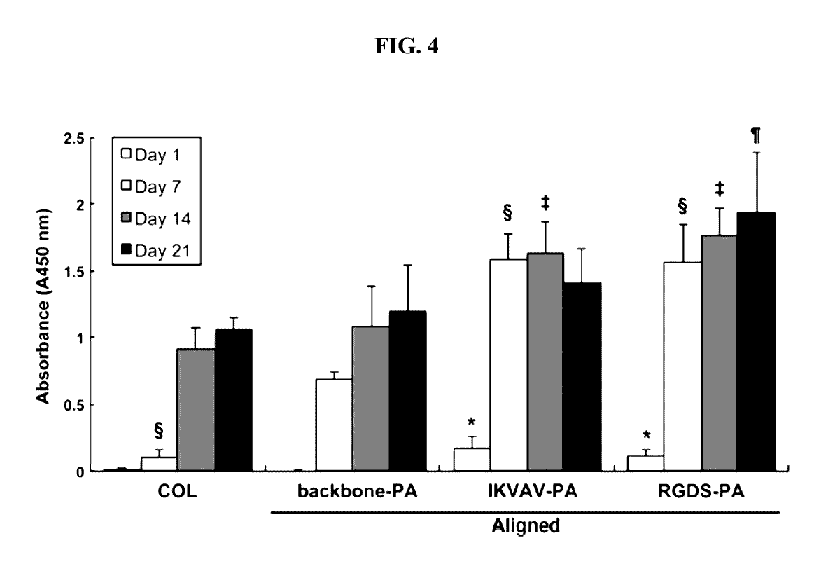

Direct observation of nanofiber alignment within the PLGA/backbone-PA construct is shown in the SEM micrograph in FIG. 3A. The vast majority of the nanofibers are oriented parallel with the tube axis, with only a few short fibers having another orientation. During sample preparation a razor blade was used to expose the gel surface, so there is the possibility that the surface fiber orientation was perturbed by the sectioning process. Therefore, the observed alignment was confirmed using birefringence and small angle x-ray scattering (SAXS). FIG. 3B shows the brightfield and birefringence microscopy of the backbone-PA gel within the construct. In this cross polarizer setup, all nanofiber domains oriented vertically and horizontally should appear dark, while diagonally oriented fiber domains should be bright. The backbone-PA area appears bright, indicating diagonal nanofiber orientation in the same direction of the tube's long axis. Further proof of the aligned nanofiber morphology can be seen in the SAXS data shown in FIG. 3C. The same sectioned sample viewed in FIG. 3A was position in the path of the X-ray beam with the long axis of the construct oriented vertically. FIG. 3Ca and b show the SAXS patterns obtained from the aligned construct and a non-aligned backbone-PA gel, respectively. The pinched profile in FIG. 3Ca reveals that the sample is very anisotropic and has a fiber alignment in the vertical direction. This alignment was obtained during fabrication when the backbone-PA fibers are sheared as they pass through the mesh screen. Without this shearing process the backbone-PA gel will have the isotropic SAXS profile shown in FIG. 3Cb.

### Schwann Cell Proliferation in PA or Collagen Gels

Schwann cells demonstrated increasing proliferation with time when cultured in collagen and in backbone and bioactive PA gels (FIG. 4). Viability measurements at day 1 demonstrated that Schwann cells proliferated more rapidly in both IKVAV-PA (SEQ ID NO: 2) and RGDS-PA (SEQ ID NO: 1) gels compared to backbone-PA gel. By 7 days, proliferation in both IKVAV-PA (SEQ ID NO: 2) and RGDS-PA (SEQ ID NO: 1) were each significantly higher than that in backbone-PA gel. Proliferation in RGDS-PA gel was the highest among all groups, continuing to grow through 21 days of culture. Growth in IKVAV-PA (SEQ ID NO: 2) was maximal at day 14, with neither significant expansion nor contraction of growth observed at day 21. Based upon these observations, RGDS-PA (SEQ ID NO: 1) was chosen for use in all subsequent in vivo experimental groups treated with bioactive PA.

### Cytoskeletal Assessment and Focal Adhesion Protein Stain

The effect of the aligned PA gels on the spatial arrangement of cytoskeletal actin was examined using actin stain (FIG. 5). Schwann cells were well-spread and randomly oriented with respect to their cytoskeletal elements in 2D culture or in a collagen gel. On the other hand, significant cytoskeletal alignment and cellular elongation were observed in aligned PA gels. There was no significant difference in actin appearance when comparing RGDS-PA (SEQ ID NO: 1) and IKVAV-PA (SEQ ID NO: 2) gels. Vinculin immunostaining was performed for Schwann cells to visualize the exact location of focal adhesion sites. Vinculin-positive sites were observed on all aligned PA gels.

Latency to Hind Paw Withdrawal from a Thermal Stimulus after Sciatic Nerve Defect Reconstruction

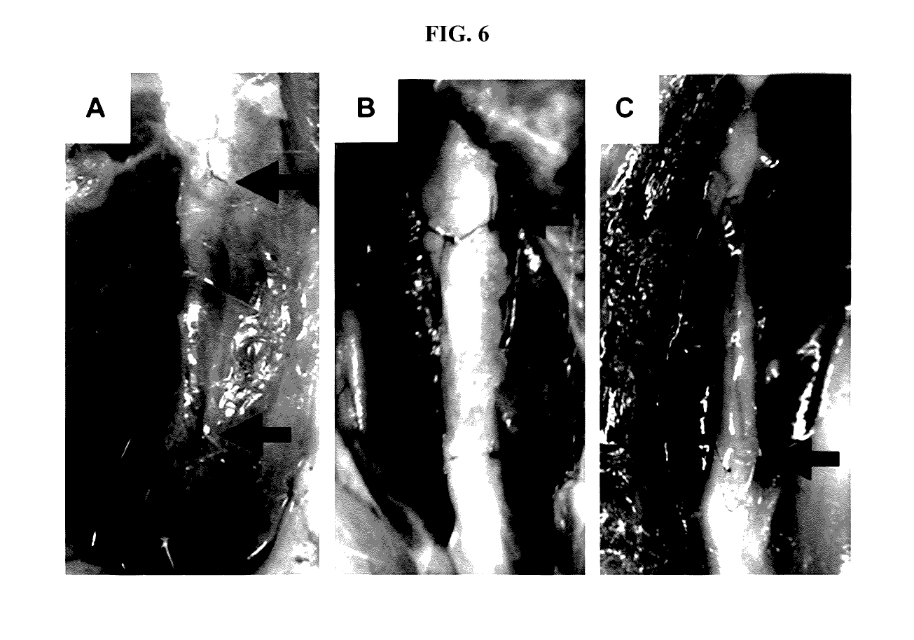

FIG. 6 shows typical gross observations of sciatic nerve in the various experimental groups 12 weeks after surgery. In the negative control group, no nerve regeneration was observed (FIG. 6A). On the other hand, nerve growth was observed in the autograft group (FIG. 6B) and PLGA/RGDS-PA (SEQ ID NO: 1) construct group (FIG. 6C). PLGA conduit was degraded completely during the postoperative period as anticipated (FIG. 6C).

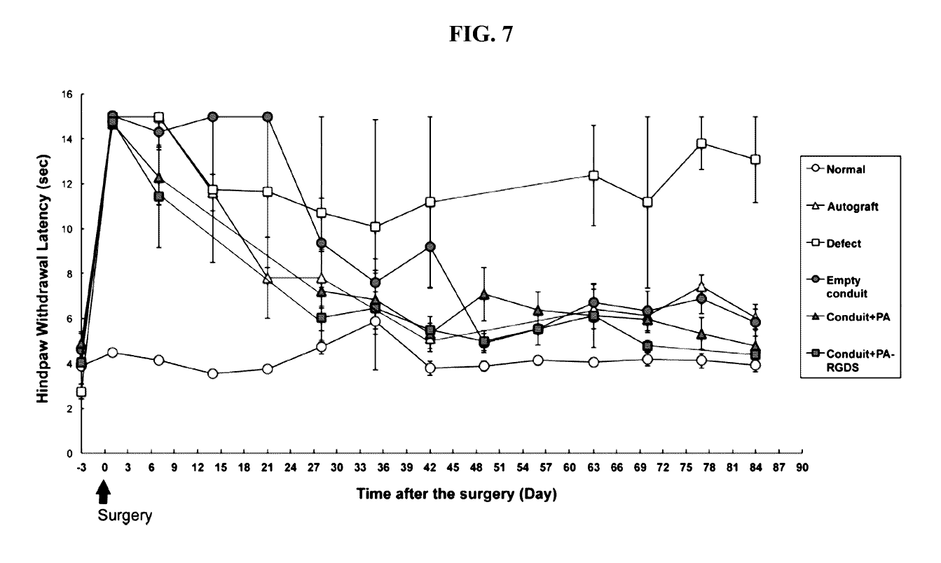

FIG. 7 shows the time profile of latency to hind paw withdrawal from a thermal stimulus after various treatments of sciatic nerve defects. The unoperated limb of each experimental animal was used as a control. The average time for the control hind paw to withdraw from a thermal stimulus measured consistently between 4 and 6 s throughout the testing period. Significant increases in reaction time were seen in all hind paws that received sciatic nerve transection on day 0. Improvement in this time was observed beginning on postoperative day 7 for the PLGA/backbone-PA and PLGA/RGDS-PA (SEQ ID NO: 1) construct groups and on day 14 for the autograft group. The empty conduit group did not show significant improvement until day 28. Ultimately, all treatment groups recovered to a baseline level of function relative to controls, except for the defect group, in which stimulus response times remained elevated.

Motor Function Recovery after Sciatic Nerve Defect Reconstruction

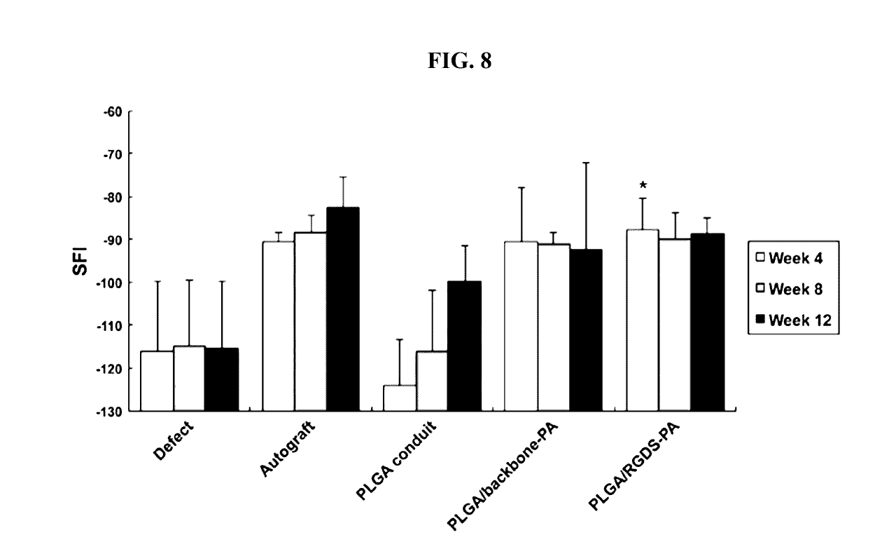

Motor function recovery was assessed using the walking track analysis and SFI. Results are shown for autologous repair, empty conduit, PLGA/backbone-PA, and PLGA/RGDS-PA (SEQ ID NO: 1) in FIG. 8. An SFI value of −100 represents complete impairment, whereas a value near 0 indicates normal motor function (ref. 25; herein incorporated by reference in its entirety). Negative control animals that received a critical sized sciatic nerve defect without any repair constantly showed SFI values of −100 throughout the entire 12 week period of testing, indicating no sciatic nerve motor recovery. Notably, PLGA/backbone-PA and PLGA/RGDS-PA (SEQ ID NO: 1) construct groups showed early improvement within 4 weeks, comparable to the autologous group. Furthermore, the SFI values in the PLGA/RGDS-PA (SEQ ID NO: 1) group showed statistically significant improvement 4 weeks after the surgery compared to that of the empty conduit group, and levels that were comparable to the autograft group.

### Histological Findings and Quantitative Analysis

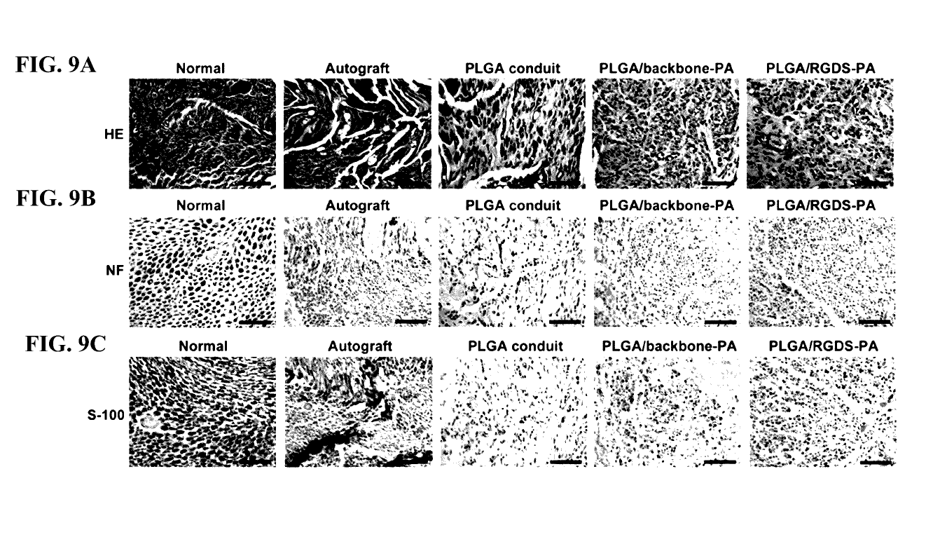

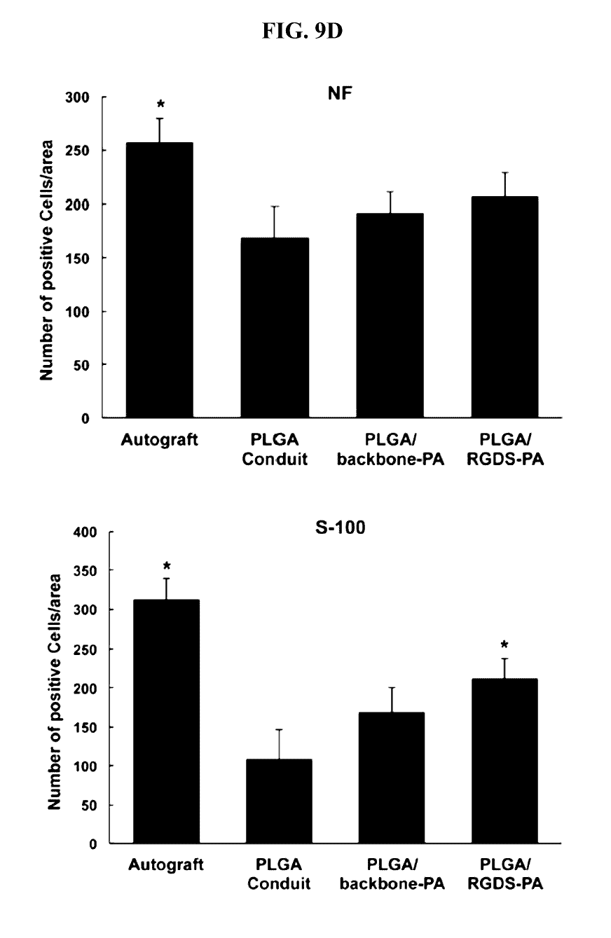

At 12 weeks, rats were euthanized and the sciatic nerve specimens were harvested. Transverse sections of the regenerated nerve in each group were stained with Hematoxylin-Eosin (FIG. 9A), anti-NF antibody to evaluate axonal regeneration (FIG. 9B), and anti-S100 antibody for Schwann cells (FIG. 9C). The density of regenerated axons in the center of the regenerate nerve was higher in both PLGA/backbone-PA and PLGA/RGDS-PA (SEQ ID NO: 1) construct groups relative to the empty PLGA conduit group. Furthermore, the axonal arrangement in these two groups was similar to that of the Autograft group. Only a few nerve fibers grew along the empty conduit, and the arrangement of the regenerated nerve fiber was attenuated in the empty tube. As shown in FIG. 9C, PLGA/backbone-PA and PLGA/RGDS-PA (SEQ ID NO: 1) construct groups exhibited S-100-positive staining cells 12 weeks after the surgery, demonstrating the regeneration and repopulation of Schwann cells within the bioengineered nerve constructs. Quantitative analysis also shows that the number of NF positive cells and S-100 positive cells in the regenerate nerve was higher in both PLGA/backbone-PA and PLGA/RGDS-PA (SEQ ID NO: 1) construct groups relative to the empty PLGA conduit group (FIG. 9D). These data suggested that the regeneration and migration of Schwann cells played a key role in the successful axonal regeneration of the critical size peripheral nerve gap.

All publications and patents mentioned above and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

The following references, some of which are cited above by number, are herein incorporated by reference in their entireties.
(1) Kehoe S, Zhang X F, Boyd D. FDA approved guidance conduits and wraps for peripheral nerve injury: a review of materials and efficacy. Injury 2012; 43:553e72.
(2) Visser P A, Hermreck A S, Pierce G E, Thomas J H, Hardin C A. Prognosis of nerve injuries incurred during acute trauma to peripheral arteries. Am J Surg 1980; 140:596e9.
(3) Millesi H. Microsurgery of peripheral nerves. Hand 1973; 5:157e60.
(4) Naff N J, Ecklund J M. History of peripheral nerve surgery techniques. Neurosurg Clin N Am 2001; 12:197e209. x.
(5) Daly W, Yao L, Zeugolis D, Windebank A, Pandit A. A biomaterials approach to peripheral nerve regeneration: bridging the peripheral nerve gap and enhancing bioactive recovery. J R Soc Interface 2012; 9:202e21.
(6) Ducic I, Fu R, Iorio M L. Innovative treatment of peripheral nerve injuries: combined reconstructive concepts. Ann Plast Surg 2012; 68:180e7.
(7) Carriel V, Alaminos M, Garzon I, Campos A, Cornelissen M. Tissue engineering of the peripheral nervous system. Expert Rev Neurother 2014; 14:301e18.
(8) Bushnell B D, McWilliams A D, Whitener G B, Messer T M. Early clinical experience with collagen nerve tubes in digital nerve repair. J Hand Surg Am 2008; 33:1081e7.
(9) Evriviades D, Jeffery S, Cubison T, Lawton G, Gill M, Mortiboy D. Shaping the military wound: issues surrounding the reconstruction of injured servicemen at the royal Centre for defence medicine. Philos Trans R Soc Lond B Biol Sci 2011; 366:219e30.
(10) Zhang S, Greenfield M A, Mata A, Palmer L C, Bitton R, Mantei J R, et al. A selfassembly pathway to aligned monodomain gels. Nat Mater 2010; 9:594e601.
(11) Berns E J, Sur S, Pan L, Goldberger J E, Suresh S, Zhang S, et al. Aligned neurite outgrowth and directed cell migration in self-assembled monodomain gels. Biomaterials 2014; 35:185e95.
(12) McClendon M T, Stupp S I. Tubular hydrogels of circumferentially aligned nanofibers to encapsulate and orient vascular cells. Biomaterials 2012; 33:5713e22.
(13) Tysseling V M, Sahni V, Pashuck E T, Birch D, Hebert A, Czeisler C, et al. Self-assembling peptide amphiphile promotes plasticity of serotonergic fibers following spinal cord injury. J Neurosci Res 2010; 88:3161e70.
(14) Silva G A, Czeisler C, Niece K L, Beniash E, Harrington D A, Kessler J A, et al. Selective differentiation of neural progenitor cells by high-epitope density nanofibers. Science 2004; 303:1352e5.
(15) Houseman B T, Mrksich M. The microenvironment of immobilized Arg-Gly-Asp peptides is an important determinant of cell adhesion. Biomaterials 2001; 22:943e55.
(16) Irvine D J, Ruzette A V, Mayes A M, Griffith L G. Nanoscale clustering of RGD peptides at surfaces using comb polymers. 2. Surface segregation of comb polymers in polylactide. Biomacromolecules 2001; 2:545e56.
(17) Irvine D J, Mayes A M, Griffith L G. Nanoscale clustering of RGD peptides at surfaces using Comb polymers. 1. Synthesis and characterization of comb thin films. Biomacromolecules 2001; 2:85e94.
(18) Jensen T W, Hu B H, Delatore S M, Garcia A S, Messersmith P B, Miller W M. Lipopeptides incorporated into supported phospholipid monolayers have high specific activity at low incorporation levels. J Am Chem Soc 2004; 126:15223e30.
(19) Massia S P, Hubbell J A. An RGD spacing of 440 nm is sufficient for integrin alpha V beta 3-mediated fibroblast spreading and 140 nm for focal contact and stress fiber formation. J Cell Biol 1991; 114:1089e100.
(20) Maheshwari G, Brown G, Lauffenburger D A, Wells A, Griffith L G. Cell adhesion and Motility depend on nanoscale RGD clustering. J Cell Sci 2000; 113(Pt10):1677e86.
(21) Rowley J A, Mooney D J. Alginate type and RGD density control myoblast phenotype. J Biomed Mater Res 2002; 60:217e23.
(22) Mooney D J, Baldwin D F, Suh N P, Vacanti J P, Langer R. Novel approach to fabricate porous sponges of poly(D,L-lactic-co-glycolic acid) without the use of organic solvents. Biomaterials 1996; 17:1417e22.
(23) Bain J R, Mackinnon S E, Hunter D A. Bioactive evaluation of complete sciatic, peroneal, and posterior tibial nerve lesions in the rat. Plast Reconstr Surg 1989; 83:129e38.
(24) Matsuka Y, Ono T, Iwase H, Mitrirattanakul S, Omoto K S, Cho T, et al. Altered ATP release and metabolism in dorsal root ganglia of neuropathic rats. Mol Pain 2008; 4:66.
(25) Kanaya F, Firrell J, Tsai T M, Breidenbach W C. Bioactive results of vascularized versus nonvascularized nerve grafting. Plast Reconstr Surg 1992; 89:924e30.
(26) Armstrong S J, Wiberg M, Terenghi G, Kingham P J. ECM molecules mediate both Schwann cell proliferation and activation to enhance neurite outgrowth. Tissue Eng 2007; 13:2863e70.
(27) Schmidt C E, Leach J B. Neural tissue engineering: strategies for repair and regeneration. Annu Rev Biomed Eng 2003; 5:293e347.
(28) de Ruiter G C, Malessy M J, Yaszemski M J, Windebank A J, Spinner R J. Designing ideal conduits for peripheral nerve repair. Neurosurg Focus 2009; 26. E5.
(29) Kim Y P, Lee G S, Kim J W, Kim M S, Ahn H S, Lim J Y, et al. Phosphate glass fibres promote neurite outgrowth and early regeneration in a peripheral nerve injury model. J Tissue Eng Regen Med 2012 (Epub ahead of print).
(30) Neal R A, Tholpady S S, Foley P L, Swami N, Ogle R C, Botchwey E A. Alignment and composition of laminin-polycaprolactone nanofiber blends enhance peripheral nerve regeneration. J Biomed Mater Res A 2011 (Epub ahead of print).
(31) Jiang X, Mi R, Hoke A, Chew S Y. Nanofibrous nerve conduit-enhanced peripheral nerve regeneration. J Tissue Eng Regen Med 2012 (Epub ahead of print).
(32) Jin J, Park M, Rengaraj an A, Zhang Q, Limburg S, Joshi S K, et al. Bioactive motor recovery after peripheral nerve repair with an aligned nanofiber tubular conduit in a rat model. Regen Med 2012; 7:799e806.
(33) Zhu Y, Wang A, Patel S, Kurpinski K, Diao E, Bao X, et al. Engineering bi-layer nanofibrous conduits for peripheral nerve regeneration. Tissue Eng Part C Methods 2011; 17:705e15.
(34) Secchi A G, Grigoriou V, Shapiro I M, Cavalcanti-Adam E A, Composto R J, Ducheyne P, et al. RGDS peptides immobilized on titanium alloy stimulate bone cell attachment, differentiation and confer resistance to apoptosis. J Biomed Mater Res A 2007; 83:577e84.
(35) Oh J H, Kim H J, Kim T I, Baek J H, Ryoo H M, Woo K M. The effects of the modulation of the fibronectin-binding capacity of fibrin by thrombin on osteoblast differentiation. Biomaterials 2012; 33:4089e99.
(36) Gunn J W, Turner S D, Mann B K. Adhesive and mechanical properties of hydrogels influence neurite extension. J Biomed Mater Res A 2005; 72:91e7.
(37) Varejao A S, Melo-Pinto P, Meek M F, Filipe V M, Bulas-Cruz J. Methods for the experimental bioactive assessment of rat sciatic nerve regeneration. Neurol Res 2004; 26:186e94.
(38) Niu Y, Chen K C, He T, Yu W, Huang S, Xu K. Scaffolds from block polyurethanes based on poly(varepsilon-caprolactone) (PCL) and poly(ethylene glycol) (PEG) for peripheral nerve regeneration. Biomaterials 2014; 35:4266e77.
(39) Castaneda F, Kinne R K. Omental graft improves bioactive recovery of transected peripheral nerve. Muscle Nerve 2002; 26:527e32.
(40) Wong B J, Mattox D E. Experimental nerve regeneration. A review. Otolaryngol Clin North Am 1991; 24:739e52.
(41) Varejao A S, Cabrita A M, Geuna S, Patricio J A, Azevedo H R, Ferreira A J, et al. Bioactive assessment of sciatic nerve recovery: biodegradable poly (DLLAepsilon-CL) nerve guide filled with fresh skeletal muscle. Microsurgery 2003; 23:346e53.
(42) Masters D B, Berde C B, Dutta S K, Griggs C T, Hu D, Kupsky W, et al. Prolonged regional nerve blockade by controlled release of local anesthetic from a biodegradable polymer matrix. Anesthesiology 1993; 79:340e6.
(43) Millan M J. The induction of pain: an integrative review. Prog Neurobiol 1999; 57. 1e164.
(44) Craig A D, Andrew D. Responses of spinothalamic lamina I neurons to repeated brief contact heat stimulation in the cat. J Neurophysiol 2002; 87:1902e14.

1. A composition comprising peptide amphiphile nanofibers aligned within a conduit.

2. The composition of claim 1, wherein the conduit comprises a polymer material.

3. The composition of claim 2, wherein the polymer material is biodegradeable.

4. The composition of claim 2, wherein the polymer material is selected from polyglycolic acid (PGA), polylactic acid (PLA), poly(lactic acid-co-glycolic acid) (PLGA), poly-ε-caprolactone (PCL), poly(diol citrate), and combinations thereof.

5. The composition of claim 1, wherein the peptide amphiphiles comprise a hydrophobic non-peptide tail, a structured peptide segment, a charged peptide segment.

6. The composition of claim 5, wherein all or a portion of the peptide amphiphiles further comprise a terminal bioactive moiety.

7. The composition of claim 1, wherein the peptide amphiphiles comprise the sequence VVAAEE (SEQ ID NO: 3).

8. The composition of claim 7, wherein the peptide amphiphiles further comprise a palmitoyl hydrophobic non-peptide tail.

9. The composition of claim 7, wherein a portion of the peptide amphiphiles further comprise a terminal bioactive moiety.

10. The composition of claim 9, wherein the terminal bioactive moiety is selected from RGDS (SEQ ID NO: 1) and IKVAV (SEQ ID NO: 2).

11. The composition of claim 1, wherein the internal diameter of the conduit is 0.5-3 mm in diameter.

12. A method of aligning peptide amphiphile (PA) nanofibers comprising extruding a PA solution through a size-limiting filter.

13. The method of claim 12, wherein the size-limiting filter is a mesh screen.

14. The method of claim 12, wherein the size-limiting filter comprises 10-100 μm pores.

15. The method of claim 12, wherein the PA solution is extruded through the mesh screen into a conduit.

16. The method of claim 12, wherein the internal diameter of the conduit is 0.5-3 mm in diameter.

17. A method of repairing or reconstructing a peripheral nerve defect comprising placing the defect in contact a composition of claim 1.

18. The use of a composition of claim 1 for tissue regeneration.
